(19) United States
(12) Patent Application Publication
(10) Pub. No.: US 2021/0247395 A1
(43) Pub. Date: Aug. 12, 2021

(54) ANTIBODY PAIRS FOR USE IN A RAPID INFLUENZA B DIAGNOSTIC TEST

(71) Applicant: GlaxoSmithKline Consumer Healthcare Holdings (US) LLC, Wilmington, DE (US)

(72) Inventors: Dennis Joseph CHURCH, Nyon (CH); Anthony NICHOLS, Nyon (CH)

(21) Appl. No.: 16/973,565

(22) PCT Filed: Jun. 11, 2019
(86) PCT No.: PCT/US2019/036463

§ 371 Date: Dec. 9, 2020

Related U.S. Application Data
(60) Provisional application No. 62/683,174, filed on Jun. 11, 2018.

(51) Int. Cl. G01N 33/569; G01N 33/558; G01N 33/68

(52) U.S. Cl. CPC G01N 33/56983; G01N 2800/52; G01N 33/68; G01N 33/558

(57) ABSTRACT

Novel antibody pairs for use in an Influenza B diagnostic test.

### SUMMARY OF THE INVENTION

The present invention is directed to novel selections of monoclonal antibody (mAb) species that provide highly sensitive immune-chromatographic assays (hereinafter, “immunoassays”) for detecting human Influenza B in a biological sample. Said mAb species are particularly useful when paired as detection and capture reagents in a lateral flow immunoassay (LFIA) of the “sandwich type,” such as is commonly utilized in so-called “Rapid Influenza Diagnostic Tests” (RIDTs) (otherwise known as “near patient” or “point-of-care” tests (POCT) or “dipsticks”, see WHO Paper, “Use of Influenza Rapid Diagnostic Tests” (2010)).

The novel mAb selections of the invention, also referred to herein as “antibody (or mAb) pairs” or “matched antibody pairs” are selected from species having binding affinity for viral Nucleoprotein (NP) (also known as Nucleocapsid protein or Protein N), a highly conserved protein across influenza B viruses which can be used to distinguish between Influenza A and B and has been a common target of marketed RIDTs (WHO, 2010 at p. 11).

While the specificity of currently available RIDT's for seasonal influenza, i.e. as against other respiratory virus and bacteria infecting the upper respiratory tract, has been high (median 90-95%), the sensitivity, however, has been highly variable (i.e. 10-96%) as compared with RT-PCR or viral culture. Low sensitivity creates a serious risk of false negative results.

We have identified certain matched antibody pairs for use as reagents in an immunoassay of the “sandwich type” which surprisingly provide a limit of detection (LOD) of live virus as low as about 10 nanogram of viral protein per milliliter of sample, which so far as we are aware, significantly improves over the sensitivity of available RIDTs.

This very low LOD was only achieved using the particular matched pairs of antibodies as, respectively, the detection and capture antibodies, and could not have been predicted based on the binding affinity of the individual antibodies alone.

Moreover, it was unexpected that such high sensitivity could be achieved with antibody pairs that bind to neighboring or even identical binding antigenic epitopes of NP, as called for by the invention.

In hindsight, with knowledge of the invention, it is hypothesized, although without intending to be bound thereby, that the steric hindrance which would normally be expected to inhibit simultaneous binding of such pairs of antibodies to NP in its monomeric form can be offset by NP oligomer formation under certain conditions to form multivalent complexes exposing additional sites for antibody binding.

The high sensitivity exhibited by the matched antibody pairs of the invention is particularly advantageous in facilitating detection of virus even in low viral titer biological fluids such as might be self-sampled by a consumer relatively non-invasively, e.g, by swabbing mucosal fluids such as nasal fluids or saliva.

Accordingly, in general the invention is directed to: matched antibody pairs for detecting Influenza B present in a sample (especially, a biological sample comprising live virus); and an immunoassay comprising said matched antibody pairs as the detection and capture reagents, especially an immunoassay of the sandwich type, and most preferably, an LFIA of the sandwich type.

In one aspect the invention comprises a matched antibody pair for use as the respective detection and capture antibodies in an LFIA to detect Nucleoprotein (NP) antigen of Influenza B present in a sample, wherein the antibodies of said matched antibody pair are independently capable of binding to an NP epitope in the region comprising sequential position amino acids 1-80 of Influenza B Nucleoprotein, as depicted in SEQ ID NO:1.

In another aspect, the invention comprises, as a novel composition of matter, arising in the performance of an LFIA of the sandwich type, a ternary complex comprising:
(a) Influenza B Nucleoprotein;
(b) at least one species of “detection antibody” conjugated with or otherwise associated with a detectible signal; and
(c) at least one species of “capture antibody” optionally immobilized on a substrate,
   wherein (b) and (c) are independently capable of binding an epitope of (a) in the region comprising amino acids 1-80 of Influenza B Nucleoprotein, to thereby form said ternary complex.

The invention also comprises a substrate comprising immunochromatographic material having immobilized thereon the ternary complex described above, via its capture antibody (c).

The invention further comprises a lateral flow immunoassay for detecting Nucleoprotein (NP) of Influenza B in a sample, comprising:

(a) a mobile phase comprising a detection antibody conjugated or otherwise associated with a detectible signal, said detection antibody being capable of binding NP to form a binary complex; and

(b) a stationary phase comprising immunochromatographic material having immobilized thereon a capture antibody, said capture antibody being capable of binding said NP of the binary complex to form a ternary complex, whereby a detectible signal generated by said ternary complex is indicative of the presence of NP in the sample,

said capture and detection antibodies being capable of binding to an NP epitope in the region comprising amino acids 1-80 of Influenza B Nucleoprotein.

The invention further comprises a method for detecting Influenza B in a sample using a lateral flow immunoassay comprising a mobile phase and a stationary phase, said method comprising

(a) introducing said sample to the mobile phase of said assay,
(b) contacting NP of Influenza B in said sample with a detection antibody that is conjugated or otherwise associated with a detectible signal to form a binary complex comprising said detection antibody and said NP in said mobile phase, and
(c) contacting said mobile phase with a stationary support comprising immunochromatographic material having immobilized thereon a capture antibody, said capture antibody being capable of binding said NP of the binary complex to form a ternary complex,
whereby a detectible signal generated by said ternary complex is indicative of the presence of NP in the sample,
wherein said capture antibody and said detection antibody are independently capable of binding an NP epitope located in the region defined by amino acids 1-80 of Influenza B Nucleoprotein.

In the various embodiments of the invention, each of the detection and capture antibodies of the antibody pairs of the invention is independently capable of binding the same or a different epitope of Influenza B Nucleoprotein. In one embodiment, at least one antibody of the pair, which may be either the capture antibody or the detection antibody, is capable of binding an epitope comprising SEQ ID NO: 2. In a preferred embodiment, at least the capture antibody is capable of binding an epitope comprising said SEQ ID NO:2. In another embodiment, both capture and detection antibodies are capable of binding an epitope comprising SEQ ID NO:2.

Additionally, the invention contemplates a method for diagnosing a patient (or a method for self-diagnosis by a consumer) afflicted with Influenza B comprising testing a biological sample obtained from the patient or consumer in a lateral flow immunoassay according to the invention, and determining the presence of Influenza B Nucleoprotein, the presence of said Nucleoprotein being indicative of Influenza B infection; and a method for monitoring the efficacy of therapeutic treatment of Influenza B in a patient by testing a biological sample from the patient in said assay, and determining the presence or absence of Influenza B Nucleoprotein, prior to and following administration of a pharmaceutical active agent for treating Influenza B.

Furthermore, the invention comprises a medical device or kit for rapid testing of Influenza B infection in a mammalian, especially human, subject, comprising an LFIA of the invention in a housing, optionally also comprising a viewing port and/or a reader device and/or instructions for use of the kit.

### BACKGROUND OF THE INVENTION

Influenza is a highly contagious epidemic to pandemic acute viral respiratory disease caused by genera “A,” “B” and “C” of the Orthomyxoviridae family. Influenza virus A and Influenza virus B are the two genera most commonly associated with the disease in humans.

In humans, influenza A and B viruses cause seasonal epidemics with winter peaks in temperate zones and year-round circulation in the tropics. Both viruses continually evolve through mutations leading to antigenic drift of certain glycoproteins. These can potentially cause a rare influenza pandemic if the novel virus spreads in a sustained manner through largely susceptible populations.

Uncomplicated seasonal influenza is associated with an incubation period of 1-4 days, followed by the acute onset of signs and symptoms including a fever 38° C., myalgia, headache, sore throat and a protracted cough. The clinical presentation of influenza can range from asymptomatic infection to fatal pneumonia. Children may present with gastrointestinal symptoms, while influenza in the elderly may present as lethargy without an elevated temperature.

In adults, viral replication and probable communicability is greatest in the first 3-5 days of illness. In young children and immunocompromised persons, it can be longer (e.g., 7-10 days in the former and weeks to months in the latter). Influenza infection rates tend to be highest in pediatric populations, while serious complications from influenza disease are more common in the elderly.

Influenza co-circulates with other respiratory pathogens; hence it is important to differentiate influenza from other respiratory diseases. Early influenza testing and diagnosis can facilitate the more timely administration of antiviral drugs, which, in general, are of clinical benefit when administered within 48 hours of the appearance of symptoms. Since not all antiviral drugs are effective against both influenza A and B, it is important that a diagnostic test be able to distinguish between the two.

Influenza viruses are enveloped, negative-sense (complementary to mRNA sequence), single-stranded RNA viruses with a segmented genome that contain eight segments of genomic viral RNA (vRNA) encoding up to 13 proteins.

The encoded proteins include polymerase basic protein 2 (PB2), polymerase basic protein 1 (PB1), PB1-F2, polymerase acidic protein (PA), hemagglutinin (HA), nucleoprotein (NP), neuraminidase (NA), the matrix protein (M1), the ion channel protein (M2), nonstructural protein 1 (NS1) and nuclear export protein/nonstructural protein 2 (NEP/NS2).

Nucleoprotein (NP), which is highly conserved across influenza viruses, is a multifunctional protein involved in many stages of influenza virus replication. It is the major viral protein found within viral ribonucleoprotein (vRNP) complexes, in which the RNA is encapsidated by nucleoprotein (NP) and associated with a polymerase complex. NP can form homo-oligomers, which wrap around genomic RNA along with the trimeric polymerase, adding a high-order structure to the vRNPs, see Sherry, L. et al., J Virol. 2014 November; 88(21): 12326-12338.

Early in infection, once released from the incoming virus particles, the vRNPs are transported into the nucleus of the host cell with involvement of the nuclear localization sequences (NLS) of NP. Later in infection, NP is found predominantly in the cytoplasm in the form of newly synthesized vRNPs, to be packaged into progeny virus particles.

Despite influenza A and B viruses belonging to separate genera of the Orthomyxoviridae family, their NP proteins in general share a relatively high level of sequence conservation. However, NP of influenza A viruses contains at least two regions that exhibit nuclear localization signal (NLS) activity which are absent from the NP of influenza B virus; and NP of influenza B viruses contains an evolutionarily conserved N-terminal 50-amino-acid extension that is absent from NP of influenza A virus, which also appears to be involved in nuclear localization, among other functions. It has also been suggested that all sequences able to act as an NLS are located within the first 80 amino acids of Influenza B NP, see Sherry, L. et al., J Virol. 2014, id.

Nucleoprotein of Influenza B has been well-studied, see, e.g., Chenavas et al., Future Microbiology, Vol. 8, No. 12, published online 22 Nov. 2013 at https://doi.org/10.2217/fmb.13.128; and the sequences of the Nucleoproteins of various strains of Influenza B virus have been accessioned, see, e.g., UniProt Accession No. P04665.

Structurally, NP, consists of a head and a body domain making up a tail loop/linker region. The RNA binding property of NP is known to involve the protruding element and flexible basic loop between the head and body domains, both having high degree of primary sequence conservation.

Both monomeric and multimeric (e.g., dimer, trimer and tetramer) forms of Influenza A or B NP have been observed, depending, e.g., on salt concentration and the size of the RNA molecule to which the NP is bound, see Labaronne, A. et al., Viruses 2016, 8, 247.

NP oligomerization is mediated by the insertion of the non-polymorphic and structurally conserved tail loop of one NP molecule to a groove of another NP.

Influenza B viruses are not divided into subtypes, but can be further broken down into lineages and strains. Currently there are two co-circulating lineages of the Influenza B virus based on the antigenic properties of the surface glycoprotein hemagglutinin. The lineages are termed B/Yamagata/16/88-like and B/Victoria/2/87-like viruses.

Identification of human influenza viral infections has been carried out with high specificity using laboratory methodology such as virus isolation in cell culture, or detection of viral RNA by reverse transcriptase-polymerase chain reaction (RT-PCR). RT-PCR assays detect both viable and non-viable influenza virus RNA and are in general more sensitive than cell culture. RT-PCR methodology, however, is unavailable in many geographic areas, or its capacity may be insufficient during a pandemic, and furthermore is unsuited for consumer use.

The influenza A (H1N1) pandemic that emerged in 2009 made evident the need for “point-of-care” influenza diagnostic tests that could be used by local medical practitioners treating affected patient populations to aid in case management and outbreak control and permit monitoring of disease spread and viral evolution. There has also been a significant unmet need among consumers for a rapid, relatively inexpensive, yet highly sensitive, in-home kit for detecting and diagnosing influenza virus.

The RIDTs widely used in physician's offices and clinics are generally direct antigen detection tests, typically indicating the presence of one or more viral antigens by a colorimetric, fluorescent or chemiluminescent signal. The term “rapid” has generally indicated the ability to produce results in about 30 minutes or less, and even 15 minutes or less, e.g. within about 10 minutes, or even within about 5 minutes. Thus, RIDT's can provide results in a clinically relevant time frame to complement the use of antiviral medications for treatment and chemoprophylaxis of influenza.

In an RIDT (as distinguished from a laboratory microtiter plate assay), antibody and analyte are typically bound to porous membranes, which react with positive samples while channeling excess fluids to a non-reactive part of the membrane.

RIDT's are most commonly configured as single-use LFIAs, also known as “strip tests”. LFIA's comprise a solid substrate, typically an immunochromatographic test strip, through which a mobile phase comprising the biological sample flows by capillary action to a reaction matrix where a detectable signal, such as a color change or color difference, is generated on the test strip to indicate the presence (or absence) of target analyte. The term “capillary action” or “capillarity” refers to the process by which a molecule is drawn across the lateral test strip due to such properties as surface tension and attraction between molecules. Typically, the test strip comprises various zones on which reagents, usually antibodies, are situated.

As used herein, the term “lateral flow” refers to capillary flow through a material in a horizontal direction, but will be understood to apply to the flow of a liquid from a point of application of the liquid to another lateral position even if, for example, the device is vertical or on an incline. Lateral flow depends upon properties of the liquid/substrate interaction (surface wetting or wicking action) and does not require application of outside forces, e.g., vacuum or pressure applications by the user.

In antibody “sandwich-type” LFIA formats, the mobile phase typically comprises the sample, liquid diluents and a detection antibody-signal conjugate, while the immobile phase typically comprises the capture antibody immobilized at the test and control lines, both detection and capture antibodies being specific for the analyte.

In one exemplary test format, the liquid biological sample is absorbed onto the sample pad, and the sample progresses by capillary action into the conjugate pad, where it rehydrates detection antibody particles labelled with a detectable moiety such as a colored label, allowing for the mixing of these particles with the absorbed liquid sample. The labelled antibody interacts with the specific analyte contained in the sample, thereby initiating the intermolecular interactions which are dependent on the affinity and avidity of the reagents. Then the binary complex of the labelled antibody and its specific analyte migrate along the strip toward the “capture” antibody immobilized in the reaction zone (typically in a line transverse, and preferably perpendicular, to the sample flow to form a “test line” across the strip), which recognizes and captures the complex, where it becomes immobilized and produces a distinct signal, for example, in the form of a colored line. This technique may be used to obtain quantitative or semi-quantitative results. Excess reagents move past the capture line or lines to an optional control line comprising a positive control that insures that all reagents are functional; and finally, the excess reagents are entrapped in the wick pad, which is designed to draw the biological sample across the membrane by capillary action and thereby maintain a lateral flow along the chromatography strip.

In another exemplary format sometimes referred to as a “dipstick” or “half-strip”, solutions or suspensions of labelled antibody and biological sample are combined under conditions to form a binary complex of the labelled antibody with the analyte, prior to application to a test strip. The chromatographic strip is “dipped into” or otherwise contacts the liquid, so that the complex of labelled antibody and target analyte is drawn across the strip as described above.

Examples of lateral flow assays of the sandwich type are described, e.g., in WO17127833; WO13132347; U.S. Patent Applications 2017/233460; 2011/0008910 and 2012/0178105, all incorporated by reference.

In the direct assay format preferred in the present invention, the color intensity of the test line is proportional to the concentration of the analyte in the sample. Thus, when analyte concentration falls below the LOD, or when analyte is absent, no test line will be visible. LFIAs also typically have a control line, indicating proper liquid flow through the strip, which will appear regardless whether or not analyte is present. Accordingly, in the typical direct assay format, two visible lines on the membrane is a positive result, while a single line in the control zone is a negative result.

An LFIA using the antibody pairs of the invention may optionally also comprise an additional reagent immobilized to the immunochromatographic material of the substrate that is capable of distinguishing between the binary complex just described and any detection antibody-signal that remains free. In one embodiment, the additional reagent has at least one epitope in common with the analyte, such as an analyte molecule, or derivative or fragment (i.e., analog) thereof, so that it is capable of specifically binding to free, i.e. uncomplexed, detection antibody. Alternatively, the immobilized molecule is not an analyte molecule or analog thereof, but nevertheless is capable of preferentially binding to uncomplexed detection antibody. For example, the reagent may be a secondary antibody, such as rabbit anti-mouse, IgG F(ab′)2, which has been adsorbed against Fc fragments and therefore reacts only with the Fab portion of IgG. Thus, when no analyte is present, the secondary antibody is able to bind to the free “Fab” binding domain of the anti-NP IgG1 or IgG2 monoclonal antibody. However, when analyte is present in the test sample, it first complexes with the “Fab” binding domain of the anti-NP IgG1 or IgG2 monoclonal antibody. The presence of the analyte renders the “Fab” binding domain unavailable for subsequent binding with the additional reagent.

Multiple analytes may be tested simultaneously under the same conditions by providing additional test lines of immobilized antibodies specific to such analytes in an array format. Conversely, multiple test lines loaded with the same antibody can be used for semi-quantitative assays.

The read-out, represented by the lines appearing with different intensities, can be assessed preferably by eye or using a reader device.

Visual verification and quantification of analyte is usually done by RANN scoring, which is a standard visual scoring system based on colorimetric intensity correlated with a score card typically consisting of five lines of defined intensity, ranging from very faint to very intense. For example, for basic assay parameter analyses, a strip reader value of 50 units (RANN 2) can be used for determining the threshold for the test line, and strip reader values of less than 100 units (RANN 2-3) can be used to denote the threshold for the control line.

The “signal” component of the detection antibody-signal conjugate may be selected from, for instance, luminescent compounds (e.g., fluorescent, phosphorescent, etc.); magnetic particles; radioactive compounds; and visual compounds (e.g., colored dye or metallic substance, such as gold); and so forth. Suitable visually detectable substances are described by, e.g., U.S. Pat. No. 5,670,381 to Jou, et al. and U.S. Pat. No. 5,252,459 to Tarcha, et al., which are incorporated by reference. Luminescent, radioactive and magnetic labeled particles will require the use of an electronic reader to assess the test result.

As is well-known in the art, the signal component may be used alone or in conjunction with a particle (sometimes referred to as a “bead” or “microbead”), that is either natural (e.g., latex) or synthetic. Although any synthetic particle may be used in the present invention, the particles are typically formed from polystyrene, butadiene styrenes, styreneacrylic-vinyl terpolymer, polymethylmethacrylate,) polyethylmethacrylate, styrene-maleic anhydride copolymer, polyvinyl acetate, polyvinylpyridine, polydivinylbenzene, polybutyleneterephthalate, acrylonitrile, vinylchloride-acrylates, and so forth, or an aldehyde, carboxyl, amino, hydroxyl, or hydrazide derivative thereof.

The size of the particles may vary. The average size (e.g., diameter) of the particles may range from about 0.1 nanometers to about 100 microns, in some embodiments, from about 1 nanometer to about 10 microns, and in some embodiments, from about 2 to about 250 nanometers.

A colored colloidal particle such as gold (red color), carbon (black), silica (blue color) or latex (blue color), is preferred. Latex or nanometer sized particles of gold are most commonly used. In practice, colloidal gold particles or gold nanoclusters having a diameter of 25-80 nm nm are preferred. Commercially available examples of suitable synthetic particles include 40 nm gold colloid supplied by BBI Solutions OEM Ltd.

In the preparation of the detection antibody-signal moiety conjugate, the antibody may be conjugated to the signal using any of a variety of well-known techniques. For example, covalent attachment of the signal particle to the antibody may be accomplished using carboxylic, amino, aldehyde, bromoacetyl, iodoacetyl, thiol, epoxy and other reactive or linking functional groups, as well as residual free radicals and radical cations, through which a protein coupling reaction may be accomplished. A surface functional group may also be incorporated as a functionalized co-monomer because the surface of the signal particle may contain a relatively high surface concentration of polar groups.

In addition, although signal particles are often functionalized after synthesis, such as with poly(thiophenol), the signal particles may be capable of direct covalent linking with a protein without the need for further modification.

In one suitable embodiment, the first step of conjugation is activation of carboxylic groups on the probe surface using carbodiimide. In the second step, the activated carboxylic acid groups are reacted with an amino group of an antibody to form an amide bond. The activation and/or antibody coupling may occur in a buffer, such as phosphate-buffered saline (PBS) (e.g., pH of 7.2) or 2-(N-morpholino) ethane sulfonic acid (MES) (e.g., pH of 5.3). The resulting signal particles may then be contacted with ethanolamine, for instance, to block any remaining activated sites. Overall, this process forms a conjugated detection probe, where the antibody is covalently attached to the signal. Besides covalent bonding, other attachment techniques, such as physical adsorption, may also be utilized. The favored method for both the preferred latex and gold particles is chemi-physical adsorption.

The immunochromatographic material of an LFIA generally comprises a porous membrane which may be made from any of a variety of materials through which the mobile phase is capable of migrating. Materials used to form the porous membrane may include, but are not limited to, natural, synthetic, or naturally occurring materials that are synthetically modified, such as polysaccharides (e.g., cellulose materials such as paper and cellulose derivatives, such as cellulose acetate and nitrocellulose); polyether sulfone; polyethylene; nylon; polyvinylidene fluoride (PVDF); polyester; polypropylene; silica; inorganic materials, such as deactivated alumina, diatomaceous earth, MgSO4, or other inorganic finely divided material uniformly dispersed in a porous polymer matrix, with polymers such as vinyl chloride, vinyl chloride-propylene copolymer, and vinyl chloride-vinyl acetate copolymer; cloth, both naturally occurring (e.g., cotton) and synthetic (e.g., nylon or rayon); porous gels, such as silica gel, agarose, dextran, and gelatin; polymeric films, such as polyacrylamide; and so forth.

The reaction zone is preferably formed from nitrocellulose and/or polyether sulfone materials. The term, “nitrocellulose” refers to nitric acid esters of cellulose, which may be nitrocellulose alone, or a mixed ester of nitric acid and other acids, such as aliphatic carboxylic acids having from 1 to 7 carbon atoms. For purposes of the present invention, the pore size of nitrocellulose is generally about 5 to about 15 μm.

The size and shape of the porous membrane may generally vary as is readily recognized by those skilled in the art. For instance, a porous membrane strip may have a length of from about 10 to about 100 millimeters, in some embodiments from about 20 to about 80 millimeters, and in some embodiments, from about 40 to about 60 millimeters. The width of the membrane strip may also range from about 0.5 to about 20 millimeters, in some embodiments from about 1 to about 15 millimeters, and in some embodiments, from about 2 to about 10 millimeters. Likewise, the thickness of the membrane strip is generally small enough to allow transmission-based detection. For example, the membrane strip may have a thickness less than about 500 micrometers, in some embodiments less than about 250 micrometers, and in some embodiments, less than about 150 micrometers.

The supporting substrate for the porous membrane may be positioned directly adjacent to the porous membrane, or one or more intervening layers may be positioned between the porous membrane and the support. Regardless, the support may generally be formed from any material able to carry the porous membrane. The support may be formed from a material that is transmissive to light, such as transparent or optically diffuse (e.g., translucent) materials. Also, it is generally desired that the support is liquid-impermeable so that fluid flowing through the membrane does not leak through the support. Examples of suitable materials for the support include, but are not limited to, glass; polymeric materials, such as polystyrene, polypropylene, polyester (e.g., Mylar® film), polybutadiene, polyvinylchloride, polyamide, polycarbonate, epoxides, methacrylates, and polymelamine; and so forth.

The support should have sufficient thickness to provide sufficient structural backing to the porous membrane, e.g., from about 100 to about 5,000 micrometers, in some embodiments from about 150 to about 2,000 micrometers, and in some embodiments, from about 250 to about 1,000 micrometers.

The porous membrane may be cast onto the support, and the resulting laminate die-cut to the desired size and shape; or alternatively, the porous membrane may be laminated to the support with, e.g., adhesive.

As illustrated in FIG. 1, a typical embodiment of an LFIA sandwich assay immunochromatographic test strip comprises communicating membranes generally comprising one or more (and typically, all) of following components:

(1) Sample pad (also referred to as an application zone)—an adsorbent pad onto which the test sample is applied, which is generally impregnated with an elution medium comprising buffer salts, surfactants and the like, to facilitate the transfer of sample to the conjugate zone;

(2) Conjugate (or reagent) pad (also referred to as a conjugate zone)—comprising one or more species of “detection antibodies”, typically labelled by chemical conjugation with a signal-bearing entity, such as a fluorescent or colored particle (e.g., colloidal gold nanoparticles or latex microspheres). The conjugate zone may be located before, within or after the sample application zone, seen in the running direction of the eluent liquid;

(3) Reaction membrane—a surface, typically of nitrocellulose or cellulose acetate, on which one more species of “capture antibodies,” also specific to the target analyte, are bound or otherwise immobilized. (The term “dry-down format” generally refers to a test strip prepared by spraying down and then drying the capture antibodies in a line that crosses the membrane to act as a capture zone or test line.)

(4) A control zone will typically also be present, containing antibodies specific for either the detection antibody conjugate or analyte. The test line(s) is(are) located after the conjugate/application zone and the control line(s) is(are) located after the test line. Together, the test line(s) and control line(s) comprise what is commonly referred to as the detection zone.

(5) Absorbent pad (also referred to as a “waste zone”)—a surface which functions as a wick or waste reservoir designed to draw the sample across the reaction membrane by capillary action, wicking excess reagent and preventing backflow of the liquid.

The above components, preferably mounted on a solid substrate as described above to afford better stability and handling, may be presented in a simple dipstick format or within a kit.

Kits of the invention generally comprise a housing (eg., a plastic casing) to fix in place the several components of the test strip and maintain their close association. The housing may also comprise a sample port and/or reaction window showing the capture and/or control zones. (A “half-dipstick”, typically used for antibody screening, may comprise the above elements (1)-(3) and optionally, (5), in the absence of a capture antibody or control line.)

In operation, a biological or other sample comprising the analyte, optionally diluted with buffer, is applied to the sample pad as shown in (A).

As the sample migrates to the conjugate pad, which may be located adjacent to, upstream or (as depicted) downstream from the sample deposition point, the sample encounters the detection antibody-signal conjugate; and the resulting analyte/detection antibody-signal complex (shown in (B)) migrates along the strip by capillary action; (C) The formed complexes continue to migrate along the strip, to where the analyte is captured by the capture antibody immobilized in lines, to form capture antibody/analyte/detection antibody ternary complexes on the test line. Excess detection antibody conjugate migrates further on the test strip until it reaches the control zone, where it binds to species-specific anti-immunoglobulin antibodies.

For further background, see, e.g., K. M. Koczula and A. Gallotta, Essays Biochem. 2016 Jun. 30; 60(1): 111-120, published online 2016 Jun. 30.

Although certain embodiments of immunoassay device configurations have been described above, it should be understood, that a device of the present invention may generally have any configuration desired, and need not contain all of the components described above. Various other device configurations, for instance, are described in U.S. Pat. No. 5,395,754 to Lambotte, et al.; U.S. Pat. No. 5,670,381 to Jou, et al.; and U.S. Pat. No. 6,194,220 to Malick, et al., which are incorporated herein in their entirety by reference thereto for all purposes.

RIDTs commonly come in three different formats—dipsticks, cassettes or cards. Dipsticks comprising single-use nitrocellulose strips are placed directly in wells or tubes containing the respiratory specimen and test kit extraction agent. Alternatively, the nitrocellulose strip can be placed inside a plastic housing (cassette) or bound to thick paper (card).

RIDT's can generally accept one or more types of respiratory specimen types such as saliva, nasal aspirates or swab, nasopharyngeal aspirates or swabs, and throat swabs. Various types of nasal or nasopharyngeal swabs are well-known to workers of skill in the art. The sample collection material may consist of bibulous material such as highly purified cotton fibers which are fixed to the plastic device by ultrasonic welding. Alternative materials may be polyester, rayon, polyamide or other fibrous polymeric materials. Compatible swabs can also be made from nylon, or calcium alginate.

The RIDT preferably comprises a kit that includes instructions for use. In the case of testing for Influenza, such instructions preferably comprise a recommendation that the specimen be collected as close to the onset of symptoms as possible, and generally not after 4-5 days, as viral shedding typically diminishes, and in adults virus is often not detectable, after 5 days (somewhat longer for children).

The time-to-results varies between tests but the majority of currently marketed influenza A/B RDTs can provide results in 5-15 minutes. In some cases, manufacturers specify the maximum reading time and/or provide a stopping solution that can be added to permit a reliable delayed reading. The kit may optionally include a reading instrument.

The clinical accuracy of an influenza diagnostic test is determined by the sensitivity and specificity of the test to detect an influenza virus infection as compared with a “gold” standard (usually culture). Sensitivity is the percentage of “true influenza cases” detected as positive by a test. Specificity is the percentage of “true non-influenza cases” detected as being negative by a test.

### BRIEF DESCRIPTION OF THE DRAWINGS

FIG. 1 is a schematic view of a lateral flow immunoassay, as previously described.

FIG. 2 shows the amino acid sequence of NP antigen of human Influenza B virus (strain B/Lee/1940), based on UniProt Accession No. P03466 (SEQ ID NO:1).

FIG. 3 shows the peptide sequence (in 1-letter code, 3-letter code, and full names) of an NP binding epitope of human Influenza B (SEQ ID No: 2), corresponding to position amino acids 31-43 of SEQ. ID. NO. 1.

FIG. 4 shows wet dipstick dose response curves obtained in Example 1, which plot mean Rann score (n=2) against concentration (ng/ml) of purified NP antigen of Influenza B (Florida/4/2006) using the indicated capture and conjugate antibody pairs.

FIG. 5 is a dose response curve obtained in Example 2, which plots mean Rann Score (n=3) against concentration (ng/ml) of purified NP antigen of Influenza B (Florida/4/2006), using Antibody Pair (A) of the examples.

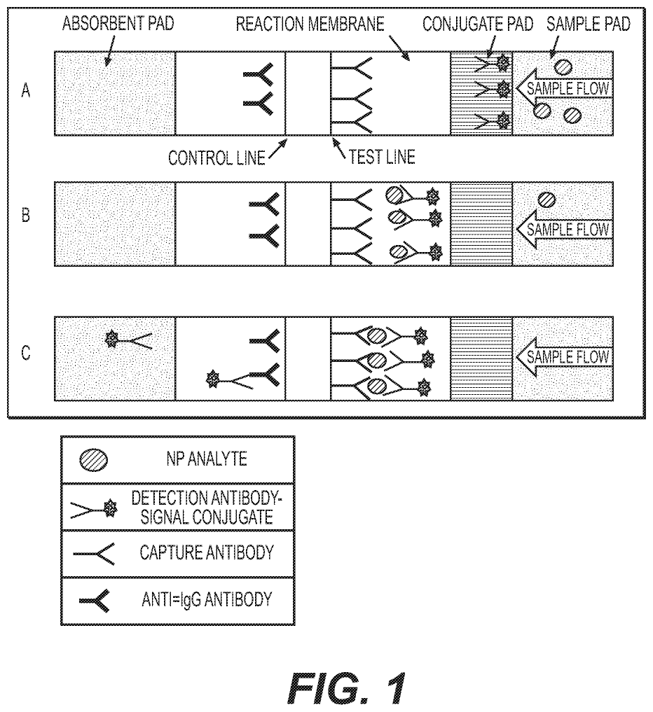

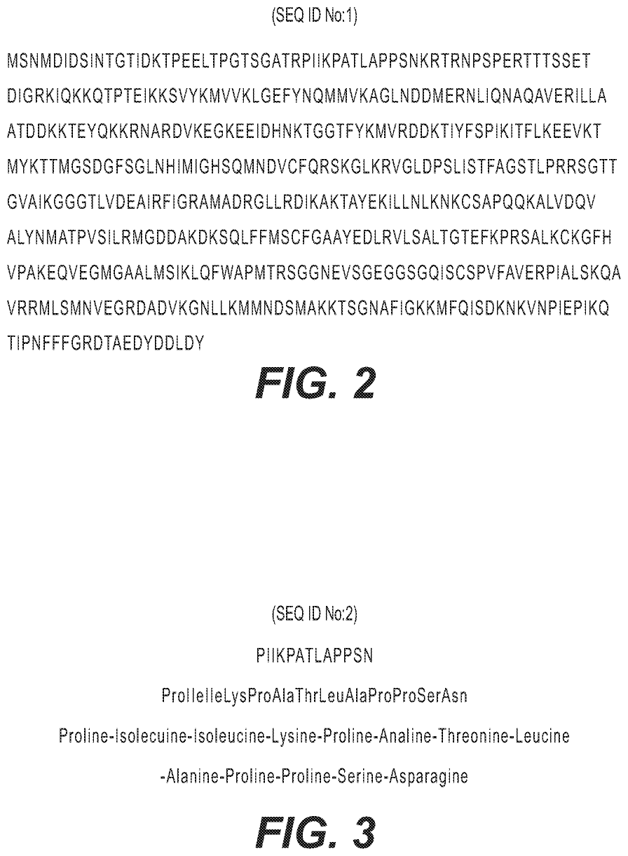

### DETAILED DESCRIPTION OF THE INVENTION

As used herein, the term “antibody” refers to immunoglobulin molecules and immunologically active portions of immunoglobulin (Ig) molecules, i.e., molecules that contain an antigen binding site that specifically binds an antigen. By “specifically binds” is meant that the antibody reacts with one or more antigenic determinants of the desired antigen and does not react with other polypeptides. Antibodies may include, but are not limited to, polyclonal, monoclonal, chimeric, dAb (domain antibody), single chain, F_{ab}, F_{ab}′ and F_{(ab)}2 fragments, scFvs, and F_{ab} expression libraries.

As used herein, the term “epitope” includes any protein determinant capable of specific binding to an antibody. Epitopic determinants usually consist of chemically active surface groupings of molecules such as amino acids or sugar side chains and usually have specific three-dimensional structural characteristics, as well as specific charge characteristics. For example, antibodies may be raised against N-terminal or C-terminal peptides of a polypeptide.

The strength, or affinity, of binding can be expressed in terms of the dissociation constant (K_{d}.) of the interaction, wherein a smaller K_{d} represents a greater affinity. Immunological binding properties of selected antibodies can be quantified using methods well known in the art. An antibody of the present invention is said to specifically bind to an influenza epitope when the equilibrium binding constant K_{d}. is ≤1 pM, preferably ≤100 nM, more preferably ≤10 nM, and most preferably ≤100 pM to about 1 pM, as measured by assays such as radioligand binding assays or similar assays known to those skilled in the art. See U.S. Pat. No. 9,951,122, which is incorporated by reference.

The term “biological sample” is intended to refer to a composition comprising tissues, cells and/or biological fluids isolated from a subject, as well as tissues, cells and/or fluids present within a subject. Examples of biological samples may comprise saliva, sputum, nasal aspirate or swab, nasopharyngeal aspirate or swab, throat swab, and cheek scraping or swab. Also included within the usage of the term “biological sample” are compositions comprising blood or a fraction or component of blood including blood serum, blood plasma, or lymph.

The novel antibody combinations of the invention comprise at least two species of antibody, at least one of which is capable of functioning as a detection antibody when conjugated or otherwise associated with a signal moiety, and at least one of which is capable of functioning as a capture antibody when bound to or otherwise immobilized on an immunochromatographic strip.

The murine monoclonal antibodies described herein are substantially homogenous; have specificity and affinity for the NP protein, and are essentially non-cross-reactive with other viral proteins.

The terms “Nucleoprotein” or “NP” as employed herein shall be understood to refer to the viral protein in its monomeric as well as its oligomeric (e.g., dimers, trimers, tetramers, etc.), especially homo-oligomeric, forms.

Certain murine anti-Influenza B nucleoprotein monoclonal antibodies are commercially available from a variety of suppliers, including Antibodies-Online, Meridian Life Science, GenWay, Fitzgerald, and Hytest, and were used to prepare the novel antibody pairs of the invention.

Said isolated and purified monoclonal antibodies can be prepared by generally known techniques, e.g., by the hybridoma method first described by Kohler et al., Nature, 256:495 (1975).

The invention is also contemplated to include NP-epitope binding fragments of said monoclonal antibodies, as well as humanized forms of said monoclonal antibodies and NP-binding fragments thereof.

NP protein binding epitopes of certain of the murine mAbs were mapped to the area close to the N-terminus of Influenza B Nucleoprotein, thus, for example, within the region comprising (e.g., consisting of one or more residues between) position amino acids 1-80; preferably amino acids 1-50; e.g., 20-50, or 25-50, even more preferably 30-45, of full-length Nucleoprotein of Influenza B.

In one embodiment, at least one of the antibodies of the combinations of the invention has affinity for, and specifically binds, the epitope disclosed as SEQ ID NO: 2, a peptide comprising residues 31-43 (PIIKPATLAPPSN) of Influenza B virus, accessioned P04665 (SEQ. ID. NO. 1).

In a preferred embodiment, at least the capture antibody has affinity for, and specifically binds, the epitope disclosed as SEQ ID NO: 2.

In a further embodiment, both capture and detection antibodies are characterized by binding affinity for SEQ. ID NO. 2.

Epitope mapping of certain of the commercially available NP monoclonal antibodies listed on Table 1 by known methods (see, e.g., H. M. Geysen et al., Proc. Natl. Acad. Sci. USA, Vol. 81, pp. 3998-4002, July 1984) using 93 overlapping peptides corresponding to the length of the NP antigen (strain B/Lee/1940) (SEQ ID NO:1), revealed that 2 of the mAbs (mAb InB12 and B265M) had an affinity and specificity (i.e. ≥0.4 OD_{492 nm} measured by ELISA) to the NP epitope sequence indicated in Table 1:

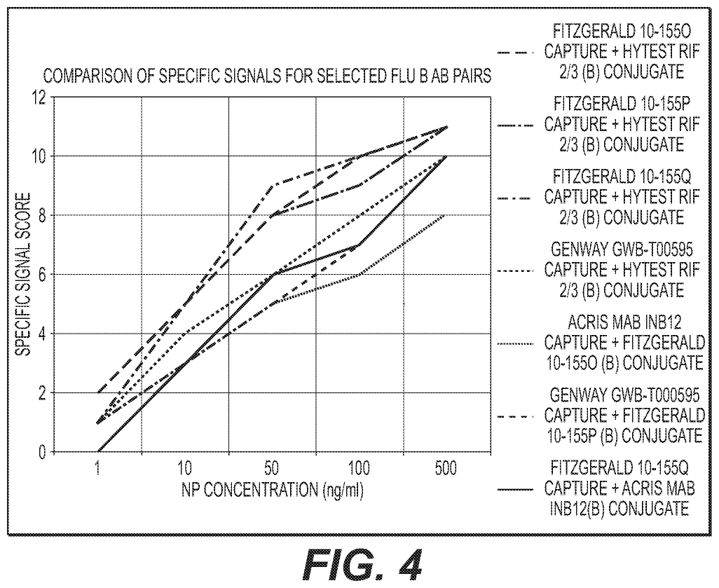

TABLE 1

| | | | | | NP Epitope | Reference in |
|---|---|---|---|---|---|---|
| Function | Vendor | Product # | Clone | Isotype | Sequence | Figure 4 |
| Capture | Acris | 3IF18-InB12 | InB12 | IgG2b | SEQ. ID. 2 | Acris Mab InB12 |
| | | | | | | cature |
| Conjugate | Acris | 3IF18-InB12 | InB12 | IgG2b | SEQ. ID. 2 | Acris Mab InB12 |
| | | | | | | (B) conjugate |
| Capture | GenWay | GWB- | B265M | IgG2b | SEQ. ID. 2 | GenWay GWB- |
| | | T00595 | | | | TOO595 capture |
| Capture/ | Fitzgerald-fii | 10-I55O | M62151 | IgG2b | | Fizgerald 10-1550 |
| Conjugate | | | | | | capture |
| Conjugate | Fitzgerald-fii | 10-155O | M62151 | IgG2b | | Fitzgerald 10-155O |
| | | | | | | (B) conjugate |
| Capture | Fitzgerald-fii | 10-I55P | M02202 | IgG1 | | Fitzgerald 10-155P |
| | | | | | | capture |
| Conjugate | Fitzgerald-fii | 10-155P | M02202 | IgG1 | | Fitzgerald 10-155P |
| | | | | | | (B) conjugate |
| Capture | Fitzgerald-fii | 10-I55Q | M12212 | IgG2b | | Fitzgerald 10-155Q |
| | | | | | | capture |
| Conjugate | Hytest | RIF17 | R2/3 | IgG2a | | Hytest RIF 2/3 (B) |
| | | | | | | conjugate |

At least one of the detection and capture antibodies of the invention has affinity for, and specifically binds, the epitope having SEQ. ID. NO:2.

In a preferred embodiment of the invention, each of the detection and capture antibodies has affinity for, and specifically binds, the epitope having SEQ. ID. NO: 2.

A preferred antibody pair of the invention comprises:

(a) as detection antibody, the mAb which is commercially available from Fitzgerald-fii as mAb 10-155P, clone M02202, preferably conjugated to colloidal gold; and

b) as capture antibody, the mAb which is commercially available from GenWay as mAb GWB-T00595, clone B265M.

The antibody pairs of the invention are particularly useful as detection and capture reagents in an LFIA of the sandwich type, as described above.

It will be evident that the term “sandwich” (or “sandwich-type”) as employed herein is intended to refer to an immunoassay in which the paired detection and capture antibodies are capable of binding to different, or the same, epitopes of optionally oligomerized NP analyte.

The following examples are provided by way of illustration only by means of various particular embodiments and are in no way to be considered limitative of the invention.

### Materials and Methods.

The following antibody pairs were tested:

TABLE 2
mAb Pairs

| | Detection | Capture |
|---|---|---|
| (A) | Hytest RIF 2/3 (B) conjugate | Fitzgerald 10-1550 capture |
| (B) | Hytest RIF 2/3 (B) conjugate | Fitzgerald 10-155P capture |
| (C) | Hytest RIF 2/3 (B) conjugate | Fitzgerald 10-155Q capture |
| (D) | Hytest RIF 2/3 (B) conjugate | GenWay GWB-TOO595 capture |
| (E) | Fitzgerald 10-1550 (B) conjugate | Acris Mab InB12 capture |
| (F) | Fitzgerald 10-155P (B) conjugate | GenWay GWB-TOO595 capture |
| (G) | Acris Mab InB12 (B) conjugate | Fitzgerald 10-155Q capture |

Detection antibody-gold conjugate. Conjugates of the detection antibody with 40 nm colloidal gold particles (HD.GC40.OD10) supplied by BBI Solutions OEM Ltd., were prepared by known techniques. The conjugates were diluted with PBS 1% Tween 20 to form a suspension having optical density (OD) of 1.
NP standards were prepared by serial dilutions of recombinant antigen of Influenza B (Florida/4/2006) in PBS 1% Tween 20 pH 7.2 to a concentration of 1-50 nanograms per milliliter.

### Example 1. Half (i.e. “Wet”) Dipstick Dose Response of Detection Antibody Conjugate to NP Antigen of Influenza B (Florida/4/2006)

Capture antibody was striped onto a nitrocellulose membrane card, 2.5×30 cm, at a rate of 0.1 microliters per second using an Isoflow reagent dispensing module. The card was allowed to dry at ambient temperature overnight in a humidity controlled dry room, and then laminated with a 21-millimeter wide cellulosic fiber wick (222 Ahlstrom membrane). The card was cut to a 5-millimeter wide strip using a kinematic slitter (Kinematic Matrix 2360), yielding 5-millimeter wide half lateral flow dipsticks (“half-sticks”).

20 μl of each of the NP stock solution and the detection antibody-gold conjugate suspension were added to a 96-well plate and incubated for 5 minutes. The half-stick samples were placed in the wells, and allowed to stand until all liquid was absorbed.

The results of visual scoring of the color intensity of the test line of the samples against a Rann scale of 0 (no visible color on the test line) to 2 (visible color on the test line) to 11 (very intense color on the test line), are shown on FIG. 4.

As shown in FIG. 4, even without optimization or amplification, the Fitzgerald 10-1550 and Hytest RIF17 2/3 antibody pair (A) can detect down to 1 ng/ml influenza B (Florida/4/2006) NP viral antigen.

### Example 2. Evaluation of Antibody Pair (F) in “Dry Down” Format

Nitrocellulose cards were striped with 1.0 mg/ml of capture antibody, and allowed to dry overnight at ambient temperature in a humidity controlled environment. A conjugate pad on the membrane card was sprayed with detection antibody colloidal gold conjugate.

Using standard techniques, devices were run with 60 μl of each of the NP standards for 20 minutes.

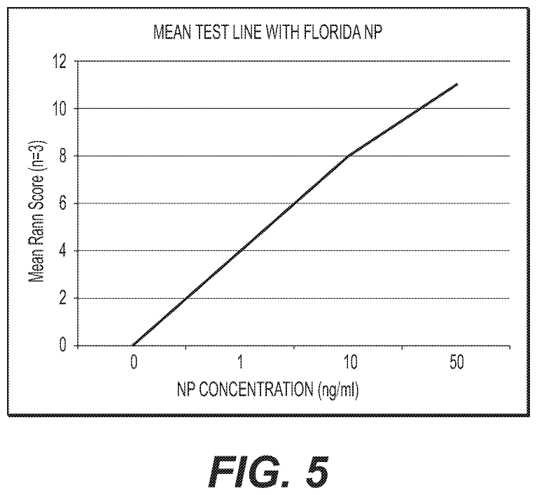

The results of visual scoring of the test line against a Rann scale as previously described are shown in FIG. 5.

Antibody Pair (F), i.e. GenWay GWB-T00595 (capture) and Fitzgerald-fii 10-155P(B) (conjugate), was shown to detect NP antigen of Influenza B (Florida/4/2006) down to 1 ng/ml.

1. A matched antibody pair comprising the detection and capture antibodies in a lateral flow immunoassay to detect Nucleoprotein (NP) antigen of Influenza B present in a sample, wherein the antibodies of said matched antibody pair are independently capable of binding to an NP epitope in the region comprising amino acids 1-80 of Influenza B Nucleoprotein.

2. A matched antibody pair according to claim 1 wherein at least one of the antibodies is capable of binding to an NP epitope comprising SEQ. ID. No. 2 hereof.

3. A matched antibody pair according to claim 1 wherein at least the capture antibody is capable of binding to an NP epitope comprising SEQ. ID. NO: 2.

4. A matched antibody pair according to claim 1 wherein the detection antibody is Fitzgerald-fii 10-I55P, clone M02202, and the capture antibody is GenWay GWB-T00595, clone B265M.

5. A ternary complex comprising:
(a) Influenza B Nucleoprotein (NP);
(b) a detection antibody conjugated with or otherwise associated with a detectible signal; and
(c) a capture antibody, optionally immobilized on a substrate,
wherein (b) and (c) are capable of binding an epitope of (a) independently selected in each case from an NP epitope in the region comprising amino acids 1-80 of Influenza B Nucleoprotein.

6. A ternary complex according to claim 5 wherein (b) and (c) are capable of binding an epitope of (a) comprising SEQ. ID. No 2 hereof, whereby said ternary complex is formed.

7. A ternary complex according to claim 5 wherein (b) and (c) are capable of binding to an epitope of (a) comprising SEQ. ID. NO: 2.

8. A ternary complex according to claim 5 wherein (b) and (c) are, respectively, Fitzgerald-fii 10-I55P, clone M02202, and the capture antibody is GenWay GWB-T00595, clone B265M.

9. A substrate comprising immunochromatographic material having immobilized thereon the ternary complex according to claim 5.

10. A lateral flow immunoassay for detecting Influenza B Nucleoprotein (NP) in a sample, comprising:
(a) a mobile phase comprising a detection antibody conjugated or otherwise associated with a detectible signal, said detection antibody being capable of binding NP to form a binary complex; and
(b) a stationary phase comprising a substrate comprising immunochromatographic material having immobilized thereon a capture antibody, said capture antibody being capable of binding said NP of the binary complex to form a ternary complex, whereby a detectible signal generated by said ternary complex is indicative of the presence of NP in the sample,
said capture and detection antibodies being capable of binding an NP epitope in the region comprising amino acids 1-80 of Influenza B Nucleoprotein.

11. A lateral flow immunoassay according to claim 10 wherein at least one of the antibodies is capable of binding to an NP epitope comprising SEQ. ID. No. 2 hereof.

12. A lateral flow immunoassay according to claim 10 wherein at least the capture antibody is capable of binding to an NP epitope comprising SEQ. ID. NO: 2.

13. A lateral flow immunoassay according to claim 10 wherein the detection antibody is Fitzgerald-fii 10455P, clone M02202, and the capture antibody is GenWay GWB-T00595, clone B265M.

14. A method for detecting Influenza B in a sample using a lateral flow immunoassay comprising a mobile phase and a stationary phase, said method comprising
(a) introducing said sample to the mobile phase of said assay,
(b) contacting Influenza B Nucleoprotein (NP) in said sample with at a detection antibody that is conjugated or otherwise associated with a detectible signal to form a binary complex comprising said detection antibody and NP in said mobile phase, and
(c) contacting said mobile phase with a stationary substrate comprising immunochromatographic material having immobilized thereon a capture antibody, said capture antibody being capable of binding said NP of the binary complex to form a ternary complex, whereby a detectible signal generated by said ternary complex is indicative of the presence of NP in the sample,
wherein said capture antibody and said detection antibody are capable of binding an NP epitope in the region comprising amino acids 1-80 of Influenza B Nucleoprotein.

15. A method for detecting Influenza B according to claim 14 wherein at least one of the antibodies is capable of binding to an NP epitope comprising SEQ. ID. No. 2 hereof.

16. A method for detecting Influenza B according to claim 14 wherein at least the capture antibody is capable of binding to an NP epitope comprising SEQ. ID. NO: 2.

17. A method for detecting Influenza B according to claim 14 wherein the detection and capture antibodies are, respectively, Fitzgerald-fii 10-I55P, clone M02202, and GenWay GWB-T00595, clone B265M.

18. A method for diagnosing a patient afflicted with Influenza B comprising testing a biological sample from the patient in a lateral flow immunoassay according to claim 10, and determining the presence of Influenza B Nucleoprotein, wherein the presence of a detectible signal is indicative of Influenza B infection in said patient.

19. (canceled)

20. (canceled)

21. (canceled)

22. A method for monitoring the efficacy of therapeutic treatment of Influenza B in a patient by testing a biological sample from the patient in a lateral flow immunoassay according to claim 10, and determining the presence or absence of Influenza B Nucleoprotein, both prior to and following administration of a pharmaceutical active agent for treating Influenza B.

23. A medical device comprising the lateral flow immunoassay according to claim 10.

24. A kit comprising the lateral flow immunoassay according to claim 10 in a housing, and optionally, a reader device and/or instructions for use of the kit.


SEQUENCE LISTING (2 sequences)

SEQ ID NO: 1 - protein, 500 aa, Influenza
MSNMDIDSINTGTIDKTPEELTPGTSGATRPIIKPATLAPPSNKRTRNPSPERTTTSSETDIGRKIQKKQTPTEIKKSVYKMVVKLGEFYNQMMVKAGLNDDMERNLIQNAQAVERILLAATDDKKTEYQKKRNARDVKEGKEEIDHNKTGGTFYKMVRDDKTIYFSPIKITFLKEEVKTMYKTTMGSDGFSGLNHIMIGHSQMNDVCFQRSKGLKRVGLDPSLISTFAGSTLPRRSGTTGVAIKGGGTLVDEAIRFIGRAMADRGLLRDIKAKTAYEKILLNLKNKCSAPQQKALVDQVALYNMATPVSILRMGDDAKDKSQLFFMSCFGAAYEDLRVLSALTGTEFKPRSALKCKGFHVPAKEQVEGMGAALMSIKLQFWAPMTRSGGNEVSGEGGSGQISCSPVFAVERPIALSKQAVRRMLSMNVEGRDADVKGNLLKMMNDSMAKKTSGNAFIGKKMFQISDKNKVNPIEIPIKQTIPNFFFGRDTAEDYDDLDY

SEQ ID NO: 2 - protein, 13 aa, Influenza
PIIKPATLAPPSN